(19) United States
(12) Patent Application Publication
(10) Pub. No.: US 2007/0004653 A1
(43) Pub. Date: Jan. 4, 2007

(54) Stable lyophilized anthracycline glycosides

(76) Inventors: Roberto Arosio, Civate (LC) (IT); Marco Villa, Milano (IT); Simonetta Amati, Milano (IT)

Correspondence Address: KENYON & KENYON LLP, ONE BROADWAY, NEW YORK, NY, 10004, US

(21) Appl. No.: 11/433,026

(22) Filed: May 11, 2006

Related U.S. Application Data
(60) Provisional application No. 60/680,139, filed on May 11, 2005.

(51) Int. Cl. A61K 31/7048

(52) U.S. Cl. 514/34

(57) ABSTRACT

The present invention provides lyophilized and stable lyophilized anthracycline glycoside salts, in particular, the hydrochloride salt. Also, the present invention provides methods of stabilizing these anthracycline glycoside salts, and methods for producing stable lyophilized anthracycline glycoside salts, such as for example the antineoplastic compound idarubicin hydrochloride, or the compounds doxorubicin hydrochloride, and epirubicin hydrochloride.

### RELATED APPLICATIONS

The present application claims the benefit of U.S. Provisional Patent Application No. 60/680,139 filed on May 11, 2005.

### FIELD OF THE INVENTION

The present invention relates to methods of preparing stable, lyophilized anthracycline glycoside hydrochlorides, in particular the hydrochloride salt of Idarubicin, Doxorubicin and Epirobicin.

### BACKGROUND OF THE INVENTION

Anthracycline glycosides are compounds having both antibiotic and anticancer activity in which a tetrahydronaphthacene chromophore is linked by a glycoside bond to a sugar, generally a basic sugar such as an amino sugar. Examples of anthracycline glycosides include doxorubicin of the formula Daunorubicin of the formula, 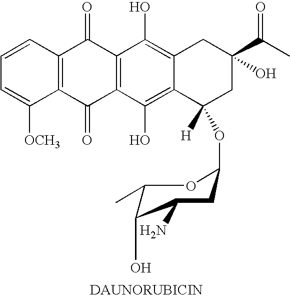
Epirubicin of the formula, 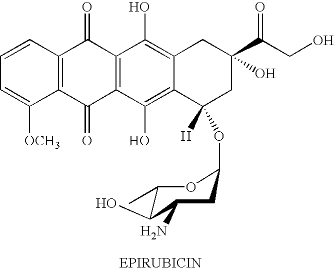
Idarubicin of the formula, 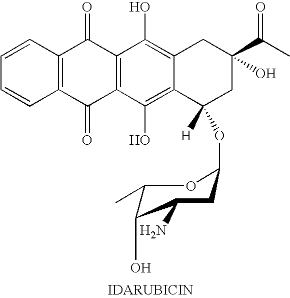
and doxorubicin of the formula,

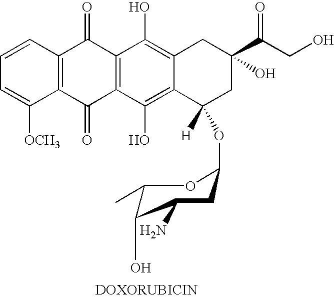

Idarubicin is the 4-demethoxy derivative of daunorubicin. Idarubicin is an antineoplastic agent that has been used to treat various cancers, including those of the breast, lung, stomach, ovaries, and lymph system. Idarubicin is marketed as an intravenous injection of Idarubicin hydrochloride of the formula, 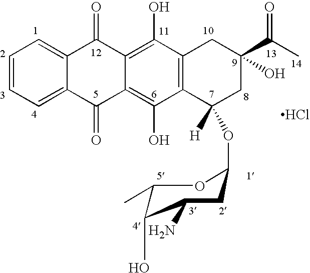
under the brand name IDAMYCIN®. Idarubicin hydrochloride is a red-orange crystalline powder, soluble in water, methanol, and other polar solvents like dimethylformamide. It is practically insoluble in acetone, chloroform, and methylene chloride. Idarubicin hydrochloride has a melting point of 175-180° C., and a pH of 5.0-6.5 in a 0.5% w/v solution in water.

Epirubicin is an antineoplastic that has been used to treat various cancers, including those of the breast, lung, stomach, ovaries, and lymph system. Epirubicin is marketed as an intravenous injection of epirubicin hydrochloride of the formula 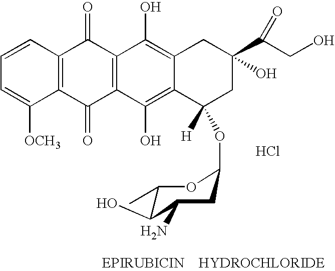
under the brand name ELLENCE® in the United States and PHARMORUBICIN® in Canada. Epirubicin is a dark red crystalline powder, soluble in water, methanol and ethyl alcohol, at a temperature of 50° C. It is practically insoluble in acetone, chloroform and in methylene chloride. Epirubicin hydrochloride has a melting point of 173-177° C., a pKa in water of 7.7, and pH of 4-5.5 in a 0.5% w/v solution in water.

Doxorubicin is an antineoplastic that has been used to treat various cancers, including those of the bladder, breast and prostate. Doxorubicin is marketed as an intravenous injection under of Doxorubicin hydrochloride of the formula 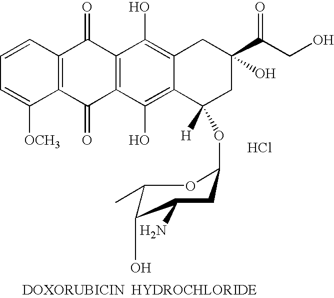
the brand name ADRIAMYCIN® in the United States and in Canada, and as ADRIBLASTIN® in Germany. Doxorubicin is a orange-red crystalline powder, soluble in water, methanol. It is practically insoluble in acetone, benzene, chloroform and ethyl ether. Doxorubicin hydrochloride has a melting point of 204-205° C.

Doxorubicin is disclosed in U.S. Pat. No. 3,590,028. A method of isolating Doxorubicin from a fermentative broth of Streptomyces peucetius var caesius is also disclosed therein.

Daunorubicin is disclosed in U.S. Pat. No. 4,012,284. A method of isolating Daunorubicin from a fermentative broth of Streptomyces peucetius is also disclosed therein.

Epirubicin is disclosed in EP patent No. 0819132. A process of obtaining Epirubicin from Daunorubicin is also disclosed therein.

Idarubicin is disclosed in DE patent No. 2525633. A process of obtaining Idarubicin from Daunomycinone is also disclosed therein.

U.S. Pat. No. 4,946,831 discloses a method of preparing a sterile, pyrogen-free, ready to use solution of anthracycline glycosides. These solutions consist of a physiologically acceptable salt of an anthracycline glycoside dissolved in a physiologically acceptable solvent, which has not been reconstituted from a lyophilizate. Also, these solutions have a pH from 2.5 to 6.5.

Since, dissolving lyophilized anthracycline glycoside salts is more facile, there is a need in the art for lyophilized anthracycline glycosides salts and for processes to prepare such lyophilized anthracycline glycosides salts.

### SUMMARY OF THE INVENTION

In one aspect, the present invention provides lyophilized anthracycline glycoside salt, wherein the anthracycline glycoside is selected from the group consisting of: Epirubicin, Idarubicin, Epidaunorubicin, Doxorubicin, and Daunorubicin. Preferably, the anthracycline glycoside is Idarubicin or Epirubicin.

In another aspect, the present invention provides a stable lyophilized anthracycline glycoside salt; wherein the anthracycline glycoside is selected from the group consisting of: Epirubicin, Idarubicin, Epidaunorubicin, Doxorubicin and Daunorubicin. Preferably, the anthracycline glycoside is Idarubicin or Epirubicin.

In yet another aspect, the present invention provides a method of stabilizing an anthracycline glycoside salt comprising combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether; wherein the anthracycline glycoside salt is selected from the group consisting of an Epirubicin salt, an Idarubicin salt, an Epidaunorubicin salt, and a Daunorubicin salt.

In yet another aspect, the present invention provides a method for stabilizing the anthracycline glycoside salt comprising combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether; and filtering the obtained mixture; wherein the resin is an organic co-polymer having a basic nature, and the anthracycline glycoside salt is selected from the group consisting of an Epirubicin salt, an Idarubicin salt, an Epidaunorubicin salt, a Doxorubicin salt and a Daunorubicin salt.

In one aspect, the present invention provides a process of purifying an anthracycline glycoside salt containing an alpha hydroxyl ketone moiety comprising combining the anthracycline glycoside salt containing an alpha hydroxyl ketone moiety with a solvent selected from the group consisting of water, and mixtures of water with alcohol, ketone or ether; heating the obtained mixture for a short period of time; and subsequently quickly cooling the obtained heated mixture.

In another aspect, the present invention provides a method of preparing stable lyophilized forms of anthracycline glycoside salts, comprising combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether; and subsequently freezing and lyophilizing the obtained mixture; wherein the anthracycline glycoside salt is selected from the group consisting of an Epirubicin salt, an Idarubicin salt, an Epidaunorubicin salt, and a Daunorubicin salt.

In another aspect, the present invention provides a method of preparing stable lyophilized forms of anthracycline glycoside salts, comprising combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether; and filtering the obtained mixture; and subsequently freezing and lyophilizing the filtrate; wherein the resin is an organic co-polymer having a basic nature, and the anthracycline glycoside salt is selected from the group consisting of an Epifubicin salt, an Idarubicin salt, an Epidaunorubicin salt, a Doxorubicin salt and a Daunorubicin salt.

In yet another aspect, the present invention provides a pharmaceutical formulation comprising a stable lyophilized anthracycline glycoside salt, preferably a hydrochloride salt, of the present invention, and pharmaceutically acceptable excipients.

In one aspect, the present invention provides a pharmaceutical formulation comprising the stable lyophilized anthracycline glycoside salt, preferably a hydrochloride salt, prepared by the processes of the present invention, and pharmaceutically acceptable excipients.

In another aspect, the present invention provides a pharmaceutical formulation comprising mixing the stable lyophilized anthracycline glycoside salt, preferably a hydrochloride salt, prepared by the processes of the present invention, and pharmaceutically acceptable excipients.

In yet another aspect, the present invention provides the use of the stable lyophilized anthracycline glycoside salt, preferably a hydrochloride salt, of the present invention for the manufacture of a pharmaceutical composition.

### DETAILED DESCRIPTION OF THE INVENTION

As used herein, the term “stable”, in reference to a lyophilized anthracycline glycoside salt, means lyophilized anthracycline glycoside salt wherein the level of the degradation products do not increase to more than a specific limit, when maintained at a specific temperature for a specific period of time.

As used herein, “room temperature” is meant to indicate a temperature of about 18-25° C.

As used herein, the term “H₂CO₃” refers to an aqueous solution of CO₂ gas.

Although dissolving a lyophilized product in water is known to be a method of choice for the rapid preparation of aqueous solutions of a drug, the claims of U.S. Pat. No. 4,946,831 expressly recite that the claimed anthracycline glycoside solutions have “not been reconstituted from a lyophilizate.” This may be because anthracycline glycosides are difficult to obtain in a stable, lyophilized form. This is especially true for Idarubicin hydrochloride, Doxorubicin hydrochloride and for Epirubicin hydrochloride.

When relating to Idarubicin hydrochloride, it is known that Idarubicin hydrochloride is not very soluble in water. The starting aqueous solution of Idarubicin hydrochloride, which is to be lyophilized, will contain about 1% w/w of Idarubicin hydrochloride, owing to its poor solubility in water. A precise stoichiometric salt is very hard to obtain during the precipitation of a highly insoluble salt like idarubicin hydrochloride. Also, a small amount of free hydrochloric acid that will generally be present in the Idarubicin hydrochloride used to prepare the solution that is to be lyophilized does not leave the product during the lyophilizing step, and instead undergoes concentration during the lyophilization process. Therefore, even a very small amount of free hydrochloric acid at the beginning of lyophilization can produce a very low local pH inside the lyophilized amorphous powder. This affects the product quality because the stability of Idarubicin hydrochloride solutions is related to the pH of the solution. However, the stability of solutions Idarubicin hydrochloride is also dependent on the storage temperature. Hence, if the solutions are not stored at proper conditions, degradation products, such as illustarated in the following scheme, are obtained

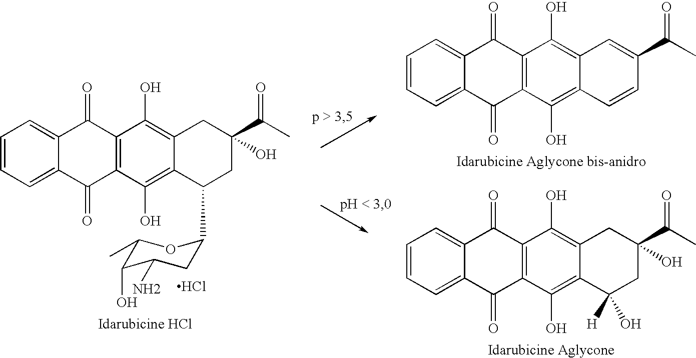

Thus, when exposing a solution of Idarubicin HCl to low pH ranges at 20° C., an increase in the content of the aglycone is detected. Further, when exposing the same solutions to the same low pH ranges at a temperature of about 4° C., the aglycone impurity is also detected, albeit in a lower amount. Moreover, when exposing a solution of Idarubicin HCl to high pH ranges an increase of the Idarubicin aglycone bis-anhydro content is detected. In addition, when exposing the same solutions to the same high pH ranges at a temperature of about 4° C., a lower amount of the bis-anhydro impurity is also detected. Furthermore, the amount of these impurities also increases, if the pH is not suitable, during the lyophilization process, thus, obtaining a product with increased content of impurites, which is not stable over time.

Therefore, Idarubicin hydrochloride was found to be most stable at pH 3.5. Idarubicin hydrochloride at this pH comprises the lowest amount of the aglycon and the bis-anhydro impurities and this amount of impurities can be considered constant over time, even at 20° C. Similarly, Doxorubicin hydrochloride and Epirubicin hydrochloride may comprise the aglycone and the bis-anhydro impurities, which amount of impurities is lowest for these compounds at the pH at which they are most stable.

Regarding the impurities in Epirubicin hydrochloride, it is known that this anthracycline glycoside exists in equilibrium with its dimer. However, the dimer content is not only dependent on the time given to equilibrate, but also on the pH.

This dimer is easily removed because it remains in the mother liquor during crystallization. However, when the crystallized product is dissolved in water to make a solution for lyophilization, a fast equilibration leads again to an increase in dimer content.

The present invention succeeds in preparing stable lyophilized anthracycline glycoside salts that are ready to be used for preparing a formulation, by stabilizing their solutions followed by a lyophilization process.

The present invention provides lyophilized anthracycline glycoside salts, wherein the anthracycline glycosides are selected from the group consisting of: Epirubicin, Idarubicin, Epidaunorubicin, and Daunorubicin. Preferably, the anthracycline glycosides are Idarubicin or Epirubicin.

The present invention also provides stable lyophilized anthracycline glycoside salts wherein the anthracycline glycosides are selected from the group consisting of: Epirubicin, Idarubicin, Epidaunorubicin, Doxorubicin and Daunorubicin. Preferably, the anthracycline glycosides are Idarubicin or Epirubicin.

A stable lyophilized Idarubicin hydrochloride of the present invention comprises lyophilized Idarubicin hydrochloride, wherein the level of the aglycon impurity does not increase by more than about 0.5% w/w, as measured by HPLC, relative to the amount of Idarubicin hydrochloride, when maintained at a temperature of about 2° C. to 8° C. for at least about 1 year, preferably for at least about 2 years, more preferably for at least about 5 years.

A stable lyophilized Doxorubicin hydrochloride of to the present invention, comprises lyophilized Doxorubicin hydrochloride, wherein the level of the aglycone impurity does not increase by more than about 2% w/w, as measured by HPLC, relative to the amount of Doxorubicin hydrochloride, when maintained at a temperature of about −20° C. for at least about 1 year.

A stable lyophilized Epirubicin hydrochloride of to the present invention comprises lyophilized Epirubicin hydrochloride, wherein the level of the dimer impurity does not increase by more than about 1% w/w, as measured by HPLC, relative to the amount of Epirubicin hydrochloride, when maintained at a temperature of about −3° C. to +5° C. for at least about 1 year, preferably for at least about 2 years, more preferably for at least about 4 years.

Preferably, the salt of the anthracycline glycoside salts is selected from the group consisting of hydrochloride (HCl), hydrogenbromide (HBr), emi-sulphate (HSO4-), and salts of organic bicarboxylic acids. Preferably, the organic bicarboxylic acid is selected from the group consisting of maleic acid, succinic acid, glutaric acid and formic acid. Preferably, the salt is a hydrochloride salt.

The present invention further provides a method of stabilizing an anthracycline glycoside salt comprising combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether; wherein the anthracycline glycoside salt is selected from the group consisting of an Epirubicin salt, an Idarubicin salt, an Epidaunorubicin salt, and a Daunorubicin salt.

The present invention provides a method for stabilizing the anthracycline glycoside salt comprising combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether; and filtering the obtained mixture; wherein the resin is an organic co-polymer having a basic nature, and the anthracycline glycoside salt is selected from the group consisting of an Epirubicin salt, an Idarubicin salt, an Epidaunorubicin salt, a Doxorubicin salt and a Daunorubicin salt.

Preferably, the anthracycline glycoside salts are selected from the group consisting of anthracycline glycoside hydrochloride, anthracycline glycoside hydrogenbromide, anthracycline glycoside emi-sulphate, and anthracycline glycoside salts from organic bicarboxylic acids such as maleic acid, succinic acid, glutaric acid, and formic acid. The anthracycline glycoside salts may contain traces of free acid, and when the salt is a hydrochloric salt, the anthracycline glycoside salt may contain traces of free hydrochloric acid.

The anthracycline glycosides salt may be obtained from the free base by dissolving the free base in an organic solvent, such as dichloromethane or chloroform, by adding the appropriate acid, for example hydrochloric acid for hydrochloric acid salts of the anthracycline glycosides, in an aqueous solution or methanol as described in GB 2.215.332 and WO 90/04601, which references are incorporated herein by reference.

Further, the solid anthracycline glycoside salt may be crystalline or amourphous. Preferably, the solid anthracycline glycoside salts is crystalline.

In the methods of stabilizing an anthracycline salt of the present invention, the alcohol solvent is preferably methanol, ethanol or isopropanol. In addition, the ketone is preferably acetone. A preferred ether in these methods is tetrahydrofuran, 1,2-dimethoxymethane or 2-methoxyethanol. Preferably, the solvent is water.

Preferably, the solid anthracycline glycoside salts are combined with the solvent to obtain a solution, prior to the addition of the buffer or the resin. Since, the solubility of the anthracycline glycoside salts is very poor, such solutions are very diluted. Preferably, the concentration of such solutions is from about 0.5% to about 5% of the anthracycline glycoside salt.

The buffer and resin are used to stabilize the anthracycline glycoside salt solutions, before lyophilizing them. The buffer may comprise a salt derived from mixing a weak base and a weak acid or from a mixture of this salt with a weak acid. Preferably, such salt is selected from the group consisting of ammonium acetate, ammonium formate, ammonium hydrogencarbonate and sodium hydrogencarbonate. Preferably, the weak acid combined with the salt is selected from the group consisting of acetic acid, formic acid and H₂CO₃. Preferred mixtures of such salt with a weak acid are selected from the group consisting of ammonium acetate and acetic acid, ammonium formate and acetic acid, ammonium formate and formic acid, ammonium hydrogencarbonate and H₂CO₃, and sodium hydrogencarbonate and H₂CO₃.

The composition of the buffer may be chosen according to the starting anthracycline glycoside salt. Preferably, a mixture of ammonium acetate and acetic acid, or a mixture of ammonium hydrogencarbonate and H₂CO₃, is used when the starting anthracycline glycoside salt is an Idarubicn salt. Preferably, when the starting anthracycline glycoside salt is an Epirubicin salt, ammnouim acetate may be used as a buffer. Preferably, ammonium acetate, a mixture of ammonium formate and acetic acid, a mixture of ammonium formate and formic acid, or a mixture of sodium hydrogencarbonate and H₂CO₃, are used as buffers when the starting anthracycline glycoside salt is a Doxorubicin salt. Ammonium hydrogencarbonate may be suitable for Doxorubicin, Idarubicin and Epirubicin salts.

Preferably, the resin may contain a tertiary amine linked to a solid surface or a salt of this teriary amine. Preferably, the salt is a weak acid salt. Preferably, the weak acid is either acetic acid or formic acid. Preferably, the resin is selected from the group consisting of: Amberlite®, Amberlite: FPA51, Amberlite FPA53, Amberlite FPA54, Amberlite FPA55, Amberlite FPA40, Amberlite FPA42, Amberlite FPA90, Amberlite FPA91, Amberlite FPA97, Amberlite FPA98, Amberlite IRA900, Amberlite IRA910, Amberjet 4200, Amberlite IRA 67, Amberlite IRA 96, Amberlyst A21, Amberlyst A23, and Amberlyst A24. More preferably, the resin is Amberlite®, most preferably, Amberlite® Ira-67. The composition of the resin may be chosen according to the starting anthracycline glycosides salt. Preferably, when the starting anthracycline glycoside salt is either a Doxorubicin or an Idarubicin salt, the resin may be AMBERLITE® IRA-67 acetate. Preferably, when the starting anthracycline glycoside salt is an Epirubicin salt, the resin may be AMBERLITE® IRA-67 free base.

The amount of buffer or resin in the methods of the present invention is in an amount of about 0.3% to about 3.0% mole equivalent per mole equivalent of the starting anthracycline glycoside salt. Preferably, the buffer and resin are used in an amount of 0.5% to about 1.5% mole equivalent per mole equivalent of the starting anthracycline glycoside salt.

After the stabilizing agent, comprising a buffer or a resin, is added the stability of the anthracycline glycoside salt at a temperature of about 4° C. to about room temperature is increased.

Preferably, when the anthracycline glycoside salts contain an alpha hydroxyl ketone moiety, a purification process is carried out, prior to stabilizing the salt. Preferably, the anthracycline glycosides salt containing an alpha hydroxyl ketone moiety is either Epirubicin or Doxorubicin.

The present invention therefore provides a process of purifying an anthracycline glycoside salt containing an alpha hydroxyl ketone moiety comprising combining the anthracycline glycoside salt containing an alpha hydroxyl ketone moiety with a solvent selected from the group consisting of water, and mixtures of water with alcohol, ketone or ether; heating the obtained mixture for a short period of time; and subsequently quickly cooling the obtained heated mixture.

Preferably, the purification is done by combining the anthracycline glycosides salt containing an alpha hydroxyl ketone moiety with water to obtain a solution. The solution is heated, preferably, to a temperature of about 50° C. to about 70° C. Preferably, the solution is heated for about 30 to about 90 minutes, more preferably, for about 30 to about 60 minutes, followed by cooling to room temperature. If the pH of the starting solution is at least 5.4, the pH may be adjusted, preferably to about 3.2 to about 3.8, by adding an acid to the solution prior to heating it. Suitable acids for adjusting the pH of the solution may include hydrogenchloride (HCl), hydrogenbromide (HBr), sulphuric acid, or bicarboxylic acids like maleic acid, succinic acid, glutaric acid and formic acid. Preferably, the acid is HCl.

The present invention provides a method of preparing stable lyophilized forms of anthracycline glycoside salts, comprising combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether; and subsequently freezing and lyophilizing the obtained mixture; wherein the anthracycline glycoside salt is selected from the group consisting of an Epirubicin salt, an Idarubicin salt, an Epidaunorubicin salt, and a Daunorubicin salt.

The present invention provides a method of preparing stable lyophilized forms of anthracycline glycoside salts, comprising combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether; and filtering the obtained mixture; and subsequently freezing and lyophilizing the filtrate; wherein the resin is an organic co-polymer having a basic nature, and the anthracycline glycoside salt is selected from the group consisting of an Epirubicin salt, an Idarubicin salt, an Epidaunorubicin salt, a Doxorubicin salt and a Daunorubicin salt.

Preferably, when using a resin as a stabilizing agent of a solution of the anthracycline glycoside salt, the resin is filtered of prior to freezing and lyophilizing the anthracycline glycoside salt. Thus, a stabilized solution just prior to lyophilization is obtained free of the stabilizing agent. As a result, the lyophilized product will also be free of the stabilizing agent.

Furthermore, in the methods of preparing stable lyophilized forms of anthracycline glycoside salts of the present invention, a purification process is preferably carried out prior to stabilizing, freezing, and lyophilizing the anthracycline glycoside salt, when the anthracycline glycoside salt contains an alpha hydroxyl ketone moiety.

The stable lyophilized anthracycline glycoside salts of the present invention contain very low amounts of aglycone and bis-anhydro degradation products. The amount of aglycone and aglycone bis-anhydro degradation products, as judged by HPLC performed by the method described in the U.S. Pharmacopeia, is less than 0.3%, preferably less than 0.2%, and even more preferably less than 0.1% aglycone degradation product, and less than 0.3%, preferably less than 0.2%, and even more preferably less than 0.15% aglycone bis-anhydro degradation product.

When the anthracycline glycoside salt contains an alpha hydroxy ketone structure, the stable lyophilized anthracycline glycoside hydrochloride contains less than 0.2%, preferably less than 0.1%.

The present invention further provides a pharmaceutical formulation comprising a stable lyophilized anthracycline glycoside salt, preferably a hydrochloride salt, of the present invention, and pharmaceutically acceptable excipients.

The present invention provides a pharmaceutical formulation comprising the stable lyophilized anthracycline glycoside salt, preferably a hydrochloride salt, prepared by the processes of the present invention, and pharmaceutically acceptable excipients.

The present invention also provides a pharmaceutical formulation comprising mixing the stable lyophilized anthracycline glycoside salt, preferably a hydrochloride salt, prepared by the processes of the present invention, and pharmaceutically acceptable excipients.

The present invention further provides the use of the stable lyophilized anthracycline glycoside salt, preferably a hydrochloride salt, of the present invention for the manufacture of a pharmaceutical composition.

As used herein, the term “pharmaceutical formulation” includes tablets, pills, powders, liquids, suspensions, emulsions, granules, capsules, suppositories, or injection preparations. The pharmaceutical composition is preferably formulated without the use of acidic excipients. Pharmaceutical compositions containing the stable lyophilized anthracycline glycoside hydrochloride of the present invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

Any excipient commonly known and used widely in the art can be used in the pharmaceutical composition. Carriers used include, but are not limited to, lactose, white sugar, sodium chloride, glucose, urea, starch, calcium carbonate, kaolin, crystalline cellulose, silicic acid, and the like. Binders used include, but are not limited to, water, ethanol, propanol, simple syrup, glucose solutions, starch solutions, gelatin solutions, carboxymethyl cellulose, shelac, methyl cellulose, potassium phosphate, polyvinylpyrrolidone, and the like. Disintegrating agents used include, but are not limited to, dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, and the like. Disintegration inhibitors used include, but are not limited to, white sugar, stearin, coconut butter, hydrogenated oils, and the like. Absorption accelerators used include, but are not limited to, quaternary ammonium base, sodium laurylsulfate, and the like. Wetting agents used include, but are not limited to, glycerin, starch, and the like. Adsorbing agents used include, but are not limited to, starch, lactose, kaolin, bentonite, colloidal silicic acid, and the like. Lubricants used include, but are not limited to, purified talc, stearates, boric acid powder, polyethylene glycol, and the like. Tablets can be further coated with commonly known coating materials such as sugar coated tablets, gelatin film coated tablets, tablets coated with enteric coatings, tablets coated with films, double layered tablets, and multi-layered tablets.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, and esters of higher alcohols, gelatin, and semisynthesized glycerides.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic.

Additional ingredients, such as dissolving agents, buffer agents, and analgesic agents may be added. If necessary, coloring agents, preservatives, perfumes, seasoning agents, sweetening agents, and other medicines may also be added to the desired preparations.

The amount of stable lyophilized anthracycline glycoside hydrochloride contained in a pharmaceutical composition for treating schizophrenia should be sufficient to treat, ameliorate, or reduce the symptoms associated with schizophrenia. Preferably, stable lyophilized anthracycline glycoside hydrochloride is present in an amount of about 1% to about 70% by weight, and more preferably from about 1% to about 30% by weight of the dose.

The pharmaceutical compositions of the invention may be administered in a variety of methods depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations may be administered intramuscularly, intracutaneously, subcutaneously or intraperitoneally. Suppositories may be administered into the rectum.

The dosage of a pharmaceutical composition for treating schizophrenia to according to the invention will depend on the method of use, the age, sex, and condition of the patient. Preferably, stable lyophilized anthracycline glycoside hydrochloride is administered in an amount from about 0.1 mg/kg to about 10 mg/kg of body weight/day. More preferably, about 1 mg to 200 mg of stable lyophilized anthracycline glycoside hydrochloride may be contained in a dose.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### Stability Tests of Idarubicin HCl

Starting product was idarubicin hydrochloride, 1% w/v aqueous solution, the analysis of which is shown in the data reported at t=0. 3% mol of NH₄OH with respect to idarubicin hydrochloride was added to the solution at t=0 and the solution was incubated at 20° C. for the indicated time. The results in table 1 show the expected rise over time of the bis-anhydro degradation product at high pH (6.69), with very little increase of aglycone.

TABLE 1

| | 20° C. | pH | % aglycone | % bis-anhydro |
|---|---|---|---|---|
| NH₃ | t = 0 | 6.69 | 0.59 | — |
| 3% mol | 25 hr | | 0.60 | 3.00 |
| | 122 hr | | 0.61 | 13.46 |

### Example 2

### Stability Tests of Idarubicin HCl

Starting product was idarubicin hydrochloride, 1% w/v aqueous solution, the analysis of which is shown in the data reported at t=0. 1% mol of NH₄OH with respect to idarubicin hydrochloride was added to the solution at t=0 and the solution was incubated at 20° C. for the indicated time.

TABLE 2

| | 20° C. | pH | % aglycone | % bis-anhydro |
|---|---|---|---|---|
| NH₃ | t = 0 | 6.56 | 0.59 | — |
| 1% mol | 29 hr | | 0.58 | 2.37 |
| | 54 hr | | 0.58 | 3.56 |
| | 146 hr | | 0.59 | 8.03 |

The results in table 2 show, again, the expected rise over time of the bis-anhydro degradation product at high pH (6.56), with very little increase of aglycone.

### Example 3

### Stability Tests of Idarubicin HCl

Starting product was idarubicin hydrochloride, 1% w/v aqueous solution, the analysis of which is shown in the data reported at t=0. 3% mol of NH₄OH with respect to idarubicin hydrochloride was added to the solution at t=0 and the solution was incubated at 4° C. for the indicated time.

These results in table 3 show that a rise over time of the bis-anhydro degradation product at high pH (6.69) occurs even at a temperature as low as 4° C.

TABLE 3

| | 4° C. | pH | % aglycone | % bis-anhydro |
|---|---|---|---|---|
| NH₃ | t = 0 | 6.69 | 0.59 | — |
| 3% mol | 28 hr | | 0.59 | 0.63 |
| | 129 hr | | 0.59 | 1.87 |

### Example 4

### Stability Tests of Idarubicin HCl

Starting product was idarubicin hydrochloride, 1% w/v aqueous solution, the analysis of which is shown in the data reported at t=0.1% mol of NH₄OH with respect to idarubicin hydrochloride was added to the solution at t=0 and the solution was incubated at 4° C. for the indicated time.

TABLE 4

| | 4° C. | pH | % aglycone | % bis-anhydro |
|---|---|---|---|---|
| NH₃ | t = 0 | 6.56 | 0.59 | — |
| 1% mol | 34 hr | | 0.58 | 0.50 |
| | 56 hr | | 0.58 | 0.74 |
| | 149 hr | | 0.58 | 1.33 |

These data confirm the results of Example 3, even at slightly lower pH.

### Example 5

### Stability Tests of Idarubicin HCl

Starting product was idarubicin hydrochloride, 1% w/v aqueous solution, the analysis of which is shown in the data reported at t=0. The solution was incubated for the indicated time at 20° C. without additional chemicals. The results in table 5 show the expected rise in bis-anhydro content.

TABLE 5

| | 20° C. | pH | % aglycone | % bis-anhydro |
|---|---|---|---|---|
| dissolved | t = 0 | 5.66 | 0.59 | — |
| as is | 26 hr | | 0.57 | 0.41 |
| | 57 hr | | 0.58 | 0.84 |
| | 148 hr | | 0.58 | 1.20 |

### Example 6

### Stability Tests of Idarubicin HCl

Starting product was idarubicin hydrochloride, 1% w/v aqueous solution, the analysis of which is shown in the data reported at t=0. The solution was incubated for the indicated time at 4° C. without the addition of additional chemicals.The results in table 6 show a rise in bis-anhydro content, even at 4° C.

TABLE 6

| | 4° C. | pH | % aglycone | % bis-anhydro |
|---|---|---|---|---|
| dissolved | t = 0 | 5.66 | 0.59 | — |
| as is | 31 hr | | 0.57 | 0.11 |
| | 154 hr | | 0.57 | 0.30 |

### Example 7

### Stability Tests of Idarubicin HCl

Starting product was idarubicin hydrochloride, 1% w/v aqueous solution. At t=0, the pH of the solution was lowered by the addition of 1% mol of HCl with respect to idarubicin hydrochloride. The solution was incubated for the indicated time at 20° C.

TABLE 7

| | 20° C. | pH | % aglycone | % bis-anhydro |
|---|---|---|---|---|
| HCl | t = 0 | 3.91 | 0.59 | — |
| 1% mol | 28 hr | | 0.57 | 0.04 |
| | 168 hr | | 0.58 | 0.09 |
| | 406 hr | | 0.59 | 0.25 |

These results show that lowerin the pH led to only 0.25% bis-anhydro, even after as long as 406 hr.

### Example 8

### Stability Tests of Idarubicin HCl

Starting product was idarubicin hydrochloride, 1% w/v aqueous solution. At t=0, the pH of the solution was lowered by the addition of 1% mol of HCl with respect to idarubicin hydrochloride. The solution was incubated for the indicated time at 4° C.

TABLE 8

| | 4° C. | pH | % aglycone | % bis-anhydro |
|---|---|---|---|---|
| HCl | t = 0 | 3.91 | 0.59 | — |
| 1% mol | 33 hr | | 0.57 | — |
| | 169 hr | | 0.58 | 0.06 |
| | 407 hr | | 0.58 | 0.05 |

The results show that lowering the pH and lowering the temperature to 4° C. led to an even smaller amount of bis-anhydro (0.05%).

### Example 9

### Stability Tests of Idarubicin HCl

Starting product was idarubicin hydrochloride, 1% w/v aqueous solution. At t=0, the pH of the solution was lowered to 3.6 by the addition of 1.3% mol of HCl with respect to idarubicin hydrochloride. The solution was then incubated for the indicated time at 20° C.

TABLE 9

| | 20° C. | pH | % aglycone | % bis-anhydro |
|---|---|---|---|---|
| As much HCl as needed | t = 0 | 3.60 | 0.59 | — |
| to pH = 3.60; | 22 hr | | 0.59 | — |
| extra HCl = 1.3% mol | 76 hr | | 0.59 | — |
| | 124 hr | | 0.60 | — |
| | 366 hr | | 0.61 | 0.01 |

The results show that lowering the pH to 3.6 led to a very small amount of bis-anhydro (0.01%).

### Example 10

### Stability Tests of Idarubicin HCl

Starting product was idarubicin hydrochloride, 1% w/v aqueous solution. At t=0, the pH of the solution was lowered to 3.55 by the addition of 1.5% mol of HCl with respect to idarubicin hydrochloride. The solution was then incubated for the indicated time at 20° C.

TABLE 10

| | 20° C. | pH | % aglycone | % bis-anhydro |
|---|---|---|---|---|
| As much HCl as needed | t = 0 | 3.55 | 0.59 | — |
| to pH = 3.55; | 22 hr | | 0.59 | — |
| extra HCl = 1.5% mol | 76 hr | | 0.59 | — |
| | 124 hr | | 0.60 | — |
| | 366 hr | | 0.61 | 0.01 |

The results again show that lowering the pH (here to 3.55) led to a very small amount of bis-anhydro (0.01%).

### Example 11

### Stability Tests of Idarubicin HCl

Starting product was idarubicin hydrochloride, 1% w/v aqueous solution. At t=0, the pH of the solution was lowered to 3.50 by the addition of 1.7% mol of HCl with respect to idarubicin hydrochloride. The solution was then incubated for the indicated time at 20° C. as shown in table 11.

TABLE 11

| | | | | % bis- |
|---|---|---|---|---|
| | 20° C. | pH | % aglycone | anhydro |
| As much HCl as needed | t = 0 | 3.50 | 0.59 | — |
| to pH = 3.50; extra HCl = 1.7% | 22 hr | | 0.59 | — |
| mol | 76 hr | | 0.59 | — |
| | 124 hr | | 0.60 | — |
| | 366 hr | | 0.61 | 0.01 |

### Example 12

### Stability Tests of Idarubicin HCl

Starting product was idarubicin hydrochloride, 1% w/v aqueous solution. At t=0, the pH of the solution was lowered to 3.03 by the addition of 6.5% mol of HCl with respect to idarubicin hydrochloride. The solution was then incubated for the indicated time at 4° C. as shown in table 12.

TABLE 12

| | | | | % bis- |
|---|---|---|---|---|
| | 4° C. | pH | % aglycone | anhydro |
| As much HCl as needed | t = 0 | 3.03 | 0.59 | — |
| to pH = 3.03; extra HCl = 6.5% | 29 hr | | 0.61 | — |
| mol | 80 hr | | 0.61 | — |
| | 364 hr | | 0.61 | — |

### Example 13

### Stability Tests of Idarubicin HCl

Starting product was idarubicin hydrochloride, 1% w/v aqueous solution. At t=0, the pH of the solution was lowered to 3.03 by the addition of 6.5% mol of HCl with respect to idarubicin hydrochloride. The solution was then incubated for the indicated time at 20° C. as shown in table 13.

TABLE 13

| | | | | % bis- |
|---|---|---|---|---|
| | 20° C. | pH | % aglycone | anhydro |
| As much HCl as needed | t = 0 | 3.03 | 0.59 | — |
| to pH = 3.03; extra HCl = 6.5% | 22 hr | | 0.62 | — |
| mol | 76 hr | | 0.63 | — |
| | 124 hr | | 0.65 | — |
| | 366 hr | | 0.77 | 0.04 |

### Example 14

### Stability Tests of Idarubicin HCl

Starting product was idarubicin hydrochloride, 1% w/v aqueous solution. At t=0, the pH of the solution was lowered to 2.18 by the addition of 48% mol of HCl with respect to idarubicin hydrochloride. The solution was then incubated for the indicated time at 4° C. as shown in table 14.

TABLE 14

| | | | | % bis- |
|---|---|---|---|---|
| | 4° C. | pH | % aglycone | anhydro |
| As much HCl as needed | t = 0 | 2.18 | 0.59 | — |
| to pH = 2.18; extra HCl = 48% | 30 hr | | 0.61 | 0.06 |
| mol | 119 hr | | 0.63 | 0.06 |

### Example 15

### Stability Tests of Idarubicin HCl

Starting product was idarubicin hydrochloride, 1% w/v aqueous solution. At t=0, the pH of the solution was lowered to 2.18 by the addition of 48% mol of HCl with respect to idarubicin hydrochloride. The solution was then incubated for the indicated time at 20° C. as shown in table 15.

TABLE 15

| | | | | % bis- |
|---|---|---|---|---|
| | 20° C. | pH | % aglycone | anhydro |
| As much HCl as needed | t = 0 | 2.18 | 0.59 | — |
| to pH = 2.18; extra HCl = 48% | 27 hr | | 0.72 | 0.06 |
| mol | 118 hr | | 1.03 | 0.06 |

### Example 16

### Stability Tests of Lyophilized Idarubicin HCl that Wasn't Stabilized

Starting product was idarubicin hydrochloride, 1% w/v in water, containing amount of free HCl. No additional chemicals were added. Most of the solution was frozen and lyophilized. A small part of the solution was diluted to 0.5% W/Vol of idarubicin hydrochloride and its pH was taken. Table 16 illustrates the increase in the amount of Idarubicin aglycone.

TABLE 16

| | pH of the | pH of the solution | | |
|---|---|---|---|---|
| | solution as | diluted to 0.5% | % | % bis- |
| | 1% w/v | w/v | aglycone | anhydro |
| Starting solid | | | 0.14 | nd |
| idarubicin | | | | |
| HCl containing a | | | | |
| trace amount of HCl | | | | |
| Solution to load into | 5.36 | 6.32 | 0.14 | nd |
| lyophilizer | | | | |
| Lyophilized product | | | 0.29 | nd |
| obtained | | | | |

To obtain idarubicin hydrochloride containing a trace amount of free HCl, idarubicin HCl was slurried in 20 volumes of a mixture of dichloromethane:methanol (90:10) containing a 1% molar amount of HCl with respect to the molar amount of idarubicin hydrochloride. The slurry was filtered off and washed with the same solvent mixture and then dried under vacuum at room temperature, until constant weight.

### Example 17

### Stability Tests of Doxorubicin HCl

Doxorubicin hydrochloride (500 mg) was dissolved in 0.001 N hydrochloric acid (20 ml) at room temperature. The pH of this solution was 3.25. The table below shows the stability data of this solution at room temperature.

TABLE 17

| Time | Temperature | % aglycone |
|---|---|---|
| 1.0 hr | 20° C. | 0.24 |
| 3.0 hr | 20° C. | 0.21 |
| 5.0 hr | 20° C. | 0.26 |
| 22.0 hr | 20° C. | 0.25 |

### Example 18

### Stability Tests of Stabilized Idarubicin HCl

Starting product was idarubicin hydrochloride, 1% w/v aqueous solution, the analysis of which is shown in the data reported at t=0. 3% mol of NH₄OAc with respect to idarubicin hydrochloride was added to the solution at t=0 and the solution was incubated at 4° C. for the indicated time.

TABLE 18

| | 4° C. | pH | % aglycone | % bis-anhydro |
|---|---|---|---|---|
| NH₄OAc | t = 0 | 5.94 | 0.59 | — |
| 3% mol | 30 hr | | 0.59 | 0.09 |
| | 131 hr | | 0.59 | 0.25 |

The results show that this treatment stabilizes the solution against formation of the aglycone.

### Example 19

### Stability Tests of Stabilized Idarubicin HCl

Starting product was idarubicin hydrochloride, 1% w/v aqueous solution, the analysis of which is shown in the data reported at t=0. 3% mol of NH₄OAc with respect to idarubicin hydrochloride was added to the solution at t=0 and the solution was incubated at 20° C. for the indicated time.

TABLE 19

| | 20° C. | pH | % aglycone | % bis-anhydro |
|---|---|---|---|---|
| NH₄OAc | t = 0 | 5.94 | 0.59 | — |
| 3% mol | 26 hr | | 0.59 | 0.50 |
| | 130 hr | | 0.59 | 1.39 |

The results show that this treatment stabilizes the solution against formation of the aglycone, even at 20° C., but the bis-anhydro content increased.

### Example 20

### Stability Tests of Stabilized Idarubicin HCl

Starting product was idarubicin hydrochloride, 1% w/v aqueous solution, the analysis of which is shown in the data reported at t=0. 1% mol of NH₄OAc with respect to idarubicin hydrochloride was added to the solution at t=0 and the solution was incubated at 4° C. for the indicated time.

TABLE 20

| | 4° C. | pH | % aglycone | % bis-anhydro |
|---|---|---|---|---|
| NH₄OAc | t = 0 | 5.92 | 0.59 | — |
| 1% mol | 24 hr | | 0.59 | 0.05 |

### Example 21

### Stability Tests of Stabilized Idarubicin HCl

Starting product was idarubicin hydrochloride, 1% w/v aqueous solution, the analysis of which is shown in the data reported at t=0. 1% mol of NH₄OAc with respect to idarubicin hydrochloride was added to the solution at t=0 and the solution was incubated at 20° C. for the indicated time.

TABLE 21

| | 20° C. | pH | % aglycone | % bis-anhydro |
|---|---|---|---|---|
| NH₄OAc | t = 0 | 5.92 | 0.59 | — |
| 1% mol | 18 hr | | 0.59 | 0.20 |
| | 313 hr | | 0.60 | 4.46 |

The results show that this treatment stabilizes the solution against formation of the aglycone, but the bis-anhydro content increased.

### Example 22

### Stability Tests of Stabilized Idarubicin HCl

Starting product was idarubicin hydrochloride, 1% w/v aqueous solution, the analysis of which is shown in the data reported at t=0. 0.5% mol of NH₄OAc and 0.5% mol of AcOH with respect to idarubicin hydrochloride were added to the solution at t=0 and the solution was incubated at 4° C. for the indicated time.

TABLE 22

| | 4° C. | pH | aglycone | bis-anhydro |
|---|---|---|---|---|
| NH₄OAc 0.5% mol + | t = 0 | 5.08 | 0.59 | — |
| AcOH 0.5% mol | 25 hr | | 0.59 | 0.04 |
| | 95 hr | | 0.59 | 0.05 |

The result shows that stability against the production of both the aglycone and the bis-anhydro degradation products can be achieved with the addition of a mixture of chemicals that has the ability to buffer the internal pH of the solution and also contains a slight acidic component.

### Example 23

### Stability Tests of Stabilized Idarubicin HCl

Starting product was idarubicin hydrochloride, 1% w/v aqueous solution, the analysis of which is shown in the data reported at t=0. 0.5% mol of NH₄OAc and 0.5% mol of AcOH with respect to idarubicin hydrochloride were added to the solution at t=0 and the solution was incubated at 20° C. for the indicated time as shown in table 30.

TABLE 23

| Idarubicin HCl | 20° C. | pH | aglycone | bis-anhydro |
|---|---|---|---|---|
| NH₄OAc 0.5% mol + | t = 0 | 5.08 | 0.59 | — |
| AcOH 0.5% mol | 19 hr | | 0.59 | 0.07 |
| | 90 hr | | 0.59 | 0.18 |

### Example 24

### Stability Tests of Stabilized Idarubicin HCl

Starting product was idarubicin hydrochloride, 1% w/v aqueous solution, the analysis of which is shown in the data reported at t=0. 1.5% mol of NH₄OAc and 1.5% mol of AcOH with respect to idarubicin hydrochloride were added to the solution at t=0 and the solution was incubated at 4° C. for the indicated time.

TABLE 24

| | 4° C. | pH | aglycone | bis-anhydro |
|---|---|---|---|---|
| NH₄OAc 1.5% mol + | t = 0 | 4.94 | 0.59 | — |
| AcOH 1.5% mol | 26 hr | | 0.59 | — |
| | 97 hr | | 0.59 | — |
| | 314 hr | | 0.59 | 0.06 |

### Example 25

### Stability Tests of Stabilized Idarubicin HCl

Starting product was idarubicin hydrochloride, 1% w/v aqueous solution, the analysis of which is shown in the data reported at t=0. 1.5% mol of NH₄OAc and 1.5% mol of AcOH with respect to idarubicin hydrochloride were added to the solution at t=0 and the solution was incubated at 20° C. for the indicated time.

TABLE 25

| | 20° C. | pH | aglycone | bis-anhydro |
|---|---|---|---|---|
| NH₄OAc 1.5% mol + | t = 0 | 4.94 | 0.59 | — |
| AcOH 1.5% mol | 21 hr | | 0.59 | 0.06 |
| | 68 hr | | 0.59 | 0.16 |
| | 139 hr | | 0.59 | 0.75 |
| | 315 hr | | 0.60 | 6.21 |

### Example 26

### Stability Tests of Stabilized Lyophilized Idarubicin HCl

Starting product was idarubicin hydrochloride: 1% w/v in water, containing a trace amount of free HCl. 1.5% mol of both NH₄OAc and AcOH with respect to idarubicin hydrochloride were added. Most of the solution was frozen and lyophilized. A small part of the solution was diluted to 0.5% W/Vol of idarubicin hydrochloride and its pH was taken.

TABLE 26

| | | pH of the | | |
|---|---|---|---|---|
| | pH of the | solution | | |
| | solution | diluted to | | % bis- |
| | as 1% w/v | 0.5% w/v | % aglycone | anhydro |
| Starting solid idarubicin | | | 0.14 | nd |
| HCl containing a trace | | | | |
| amount of HCl | | | | |
| Solution + 1.5% mol of | 5.14 | 5.26 | 0.14 | — |
| both NH₄OAc/AcOH to | | | | |
| load into lyophilizer | | | | |
| Lyophilized product | | | 0.16 | — |
| obtained | | | | |

Comparing these results to the results of Example 16 shows that the addition of a buffer and a slight acidic component reduces the amount of aglycone formed during lyophilization.

### Example 27

### Stability Tests of Stabilized Lyophilized Idarubicin HCl

Starting product was idarubicin hydrochloride, 1% w/v in water, containing a trace amount of free HCl. The solution was first decolorized with charcoal and 1.5% mol of both NH₄OAc and AcOH with respect to idarubicin hydrochloride were added. Most of the solution was frozen and lyophilized. A small part of the solution was diluted to 0.5% W/Vol of idarubicin hydrochloride and its pH was taken.

TABLE 27

| | | pH of the | | |
|---|---|---|---|---|
| | pH of the | solution | | |
| | solution | diluted to | | % bis- |
| | as 1% w/v | 0.5% w/v | % aglycone | anhydro |
| Starting solid idarubicin | | | 0.14 | nd |
| HCl containing a trace | | | | |
| amount of HCl | | | | |
| Solution decolorized with | 5.85 | 6.05 | 0.03 | — |
| charcoal + 1.5% mol of | | | | |
| both NH₄OAc/AcOH to | | | | |
| load into lyophilizer | | | | |
| Lyophilized product | | | 0.04 | — |
| obtained | | | | |

The addition of charcoal to the treatement with a buffer and a slight acidic component resulted in even further reduction in the amount of aglycone in the lyophilized product.

### Example 28

### Stability Tests of Stabilized Lyophilized Idarubicin HCl

Starting product was idarubicin hydrochloride, 1% w/v in water, containing a trace amount of free HCl. 0.65% mol of both NH₄OAc and AcOH (0.1% and 0.08% w/w, respectively) with respect to idarubicin hydrochloride were added. Most of the solution was frozen and lyophilized. A small part of the solution was diluted to 0.5% W/Vol of idarubicin hydrochloride and its pH was taken.

TABLE 28

| | | pH of the | | |
|---|---|---|---|---|
| | pH of the | solution | | |
| | solution | diluted to | | % bis- |
| | as 1% w/v | 0.5% w/v | % aglycone | anhydro |
| Starting solid idarubicin | | | 0.14 | nd |
| HCl containing a trace | | | | |
| amount of HCl | | | | |
| Solution + 0.65% mol of | | 5.72 | 0.15 | — |
| both NH₄OAc/AcOH to | | | | |
| load into lyophilizer | | | | |
| Lyophilized product | | | 0.19 | — |
| obtained | | | | |

The results show that even as little as 0.65% mol of buffer and acidic component are useful in minimizing the amount of aglycone in the lyophilized product.

### Example 29

### Stability Tests of Stabilized Lyophilized Idarubicin HCl

Starting product was idarubicin hydrochloride, 1% w/v in water, containing a trace amount of free HCl. 0.1% w/w of NH₄HCO₃ and gaseous CO₂ were added. Most of the solution was frozen and lyophilized. A small part of the solution was diluted to 0.5% W/Vol of idarubicin hydrochloride and its pH was taken.

TABLE 29

| | | pH of the | | |
|---|---|---|---|---|
| | pH of the | solution | | |
| | solution | diluted to | | % bis- |
| | as 1% w/v | 0.5% w/v | % aglycone | anhydro |
| Starting solid idarubicin | | | 0.14 | nd |
| HCl containing a trace | | | | |
| amount of HCl | | | | |
| Solution + 0.1% w/w of | | 5.73 | 0.15 | 0.06 |
| NH₄HCO₃ and gaseous | | | | |
| CO₂ needed to saturation | | | | |
| to load into lyophilizer | | | | |
| Lyophilized product | | | 0.16 | 0.05 |
| obtained | | | | |

The results show that the combination of NH₄HCO₃ buffer and CO₂ acidic component is also useful in reducing the amount of aglycone in the lyophilized product.

### Example 30

### Stability Tests of Stabilized Lyophilized Idarubicin HCl

Starting product was idarubicin hydrochloride, 1% w/v in water, containing a trace amount of free HCl. 0.1% w/w of NaHCO₃ and gaseous CO₂ were added. Most of the solution was frozen and lyophilized. A small part of the solution was diluted to 0.5% W/Vol of idarubicin hydrochloride and its pH was taken.

TABLE 30

| | | pH of the | | |
|---|---|---|---|---|
| | pH of the | solution | | |
| | solution | diluted to | | % bis- |
| | as 1% w/v | 0.5% w/v | % aglycone | anhydro |
| Starting solid idarubicin | | | 0.14 | nd |
| HCl containing a trace | | | | |
| amount of HCl | | | | |
| Solution + 0.1% w/w of | | 6.14 | 0.15 | 0.05 |
| NaHCO₃ and gaseous | | | | |
| CO₂ needed to saturation | | | | |
| to load into Lyophilizator | | | | |
| Lyophilized product | | | 0.16 | 0.05 |
| obtained | | | | |

### Example 31

### Stability Tests of Stabilized Lyophilized Idarubicin HCl

Starting product was idarubicin hydrochloride, 1% w/v in water, containing a trace amount of free HCl. 1.0% molar amount of AMBERLITE® IRA-67 (1.6 M) acetate and 0.8% molar amount of acetic acid with respect to idarubicin hydrochloride were added. The solution was filtered through a 0.4 micron membrane then it was frozen and lyophilized.

The results in table 31 show that a small amount of resin can act as well as the ammonium acetate buffer in minimizing the amount of aglycone in the lyophilized product.

TABLE 31

| | pH of the | | |
|---|---|---|---|
| | solution after | | |
| | dilution to | % | % bis- |
| | 0.5% w/v | aglycone | anhydro |
| Starting solid idarubicin HCl | | 0.14 | nd |
| containing a trace amount of HCl | | | |
| Solution + 1.0% mol of IRA-67 | 5.82 | 0.15 | — |
| acetate and 0.8% mol of acetic acid, | | | |
| slurried 2 hr, filtered, and loaded | | | |
| into lyophilizer | | | |
| Lyophilized product obtained | | 0.18 | — |

### Example 32

### Stability Tests of Stabilized Lyophilized Doxorubicin HCl

Starting product was doxorubicin hydrochloride, 2.5% w/v in water. The pH of this solution was corrected to 4.6-4.8 with IN HCl. Then 1.5% molar amount of AMBERLITE® IRA-67 (1.6 M) acetate with respect to doxorubicin hydrochloride was added. After 2 hr of slurrying, the solution was filtered through a 0.4 micron membrane then it was, frozen and lyophilized.

The results in table 32 show that a small amount of salified resin used in the preparation of doxorubicin can act as well as in the idarubicin case.

TABLE 32

| | pH of the | |
|---|---|---|
| | solution | |
| | before resin | |
| | treatment | % aglycone |
| Starting solid | 4.75 | 0.07 |
| doxorubicin | | |
| hydrochloride after | | |
| correcting pH | | |
| Solution after treatment | | 0.07 |
| with 1.5% mol of IRA-67 | | |
| acetate, slurryied 2 hr, | | |
| filtered, and loaded into | | |
| lyophilizer | | |
| Lyophilized product | | 0.09 |
| obtained | | |

### Example 33

### Stability Tests of Stabilized Doxorubicin HCl

Doxorubicin hydrochloride (500 mg) was dissolved in 0.1 N acetic acid (20 ml) at room temperature. The pH of this solution was 2.62. After 1 hour, ammonium acetate (4% molar amount with respect to the molar amount of the doxorubicin hydrochloride) was added to the solution and the solution kept at room temperature. The pH rose from 2.62 to 3.02. The results in table 46 show the stability data of this solution at room temperature. These results show that buffering at lower pH is useful.

TABLE 33

| | | % aglycone in |
|---|---|---|
| Time - Temperature | pH | doxorubicin HCl |
| 1.0 hr - 20° C. | 2.62 | 0.17 |
| addition of NH₄OAc | | |
| 0.5 hr - 20° C. | 3.02 | 0.20 |
| 1.0 hr - 20° C. | 3.02 | 0.13 |
| 1.5 hr - 20° C. | 3.02 | 0.12 |
| 2.0 hr - 20° C. | 3.02 | 0.35 |
| 3.0 hr - 20° C. | 3.02 | 0.12 |
| 6.0 hr - 20° C. | 3.02 | 0.16 |

### Example 34

### Stability Tests of Stabilized Doxorubicin HCl

Doxorubicin hydrochloride (500 mg) was dissolved in 0.05 M formic acid (20 ml) at room temperature. The pH of this solution was 2.52. After 1 hour, ammonium acetate (4% molar amount in respect of the molar amount of doxorubicin hydrochloride) was added and the solution kept at room temperature. The pH rose from 2.52 to 2.71. The table below shows the stability data of this solution at room temperature.

TABLE 34

| Time - Temperature | pH | % aglycone |
|---|---|---|
| 1.0 hr - 20° C. | 2.52 | 0.20 |
| addition of NH₄OAc | | |
| 0.5 hr - 20° C. | 2.71 | 0.13 |
| 1.0 hr - 20° C. | 2.71 | 0.11 |
| 1.5 hr - 20° C. | 2.71 | 0.12 |
| 2.0 hr - 20° C. | 2.71 | 0.10 |
| 4.0 hr - 20° C. | 2.71 | 0.14 |

These conditions lead to an aglycone content well below the acceptable limit.

### Example 35

### Purification of Doxorubicin HCl

Doxorubicin hydrochloride (500 mg) was dissolved in water (10 ml) at room temperature. A small amount of 0.1 N hydrochloric acid was added to lower the pH from 5.4 to 3.5. The solution was warmed to 60° C. for two hours and then cooled to room temperature.

TABLE 35

| Time | Temperature | % aglycone |
|---|---|---|
| t = 0 | 20° C. | nd |
| 1 hr | 60° C. | 0.11 |
| 2 hr | 60° C. | 0.31 |
| 1 hr | 20° C. | 0.21 |
| 3 hr | 20° C. | 0.22 |

### Example 36

### Purification of Epirubicin HCl

Epirubicin hydrochloride (1550 mg) (containing epirubicin aglycone 0.04% and dimer 0.8%) was dissolved in water (100 ml) and the pH was corrected to 3.2-3.8 with hydrochloric acid at room temperature. The solution was then warmed in about ½ hour to 60° C.-65° C. and then maintained at tis temperature for 1 hour and then the solution was cooled to 50-60° C. At the same time, the pH was corrected to 4.5-5.0 with sodium hydroxide (at this point epirubicin aglycone was about 0,10% and dimer content<0,10%). Then the solution was quickly transferred to a cold container.

TABLE 36

| | | | % dimer | % |
|---|---|---|---|---|
| Time | pH | Temperature | content | aglycone |
| t = 0 | 3.6 | 20° C. | 0.80 | 0.04 |
| T = 0 | | 60° C. | 0.74 | 0.04 |
| 15 min | | 60° C. | 0.38 | 0.06 |
| 30 min | | 60° C. | 0.18 | 0.07 |
| 45 min | | 60° C. | 0.11 | 0.09 |
| 60 min | | 60° C. | 0.08 | 0.11 |
| final | 4.8 | 10° C. | 0.08 | 0.11 |

### Example 37

### Purification of Epirubicin HCl

Epirubicin hydrochloride (1550 mg) (containing epirubicin aglycone 0.04% and dimer 0.8%) was dissolved in water (100 ml) and the pH corrected to 3.2-3.8 with hydrochloric acid at room temperature (by means of 30 microliters of a 1 M aqueous solution). The solution was quickly warmed (in less than ½ hour) to 60° C.-65° C. and then maintained at this temperature for 45 minutes and then the solution was cooled to 50-60° C. At the same time, the pH was corrected to 4.5-5.0 with AMBERLITE® IRA-67 free base (0,18 ml of resin 1,6M) (at this point epirubicin aglycone was about 0,09% and dimer content<0,10%). Then the solution was quickly filtered and then transferred into a cold container.

TABKE 47

| | | | % dimer | |
|---|---|---|---|---|
| Time | pH | Temperature | content | % aglycone |
| t = 0 | 3.6 | 20° C. | 0.80 | 0.04 |
| T = 0 | | 60° C. | 0.76 | 0.05 |
| 10 min | | 60° C. | 0.38 | 0.05 |
| 20 min | | 60° C. | 0.18 | 0.07 |
| 30 min | | 60° C. | 0.11 | 0.09 |
| 40 min | | 60° C. | 0.08 | 0.10 |
| Final | 4.7 | 10° C. | 0.08 | 0.10 |

### Example 38

### Preparation of Stable Lyophilized Idarubicin HCl

Idarubicin hydrochloride (500 mg) was dissolved in water (50 ml) and acetic acid and ammonium acetate (both 1.5% molar amount with respect to the molar amount of idarubicin hydrochloride) were added. The solution was frozen and lyophilized. The lyophilized idarubicin hydrochloride obtained (490 mg) showed 99.8% purity (containing 0.12% aglycone).

### Example 39

### Preparation of Stable Lyophilized Idarubicin HCl

Idarubicin hydrochloride (500 mg) was dissolved in water (50 ml) and acetic acid and ammonium acetate (both 1.5% molar amount with respect to the molar amount of idarubicin hydrochloride) were added. Charcoal was then added (100 g) and the solution was stirred for two hours. After that, the suspension was filtered through 0.4 micron membrane and then the solution was frozen and lyophilized. The lyophilized idarubicin hydrochloride obtained (490 mg) showed 99.9% purity (containing 0.02% aglycone).

### Example 40

### Preparation of Stable Lyophilized Doxorubicin HCl

Doxorubicin hydrochloride (500 mg) was dissolved in water (20 ml) and the pH corrected to 3.5 with acetic acid at room temperature. The solution was then warmed to 60° C. for 2 hours and then ammonium acetate (1.5% molar amount with respect to the molar amount of doxorubicin hydrochloride) was added. Then the solution was quickly frozen and lyophilized. The lyophilized doxorubicin hydrochloride obtained (490 mg) showed 99.7% purity (containing 0.12% aglycone).

### Example 41

### Preparation of Stable Lyophilized Epirubicin HCl

Epirubicin hydrochloride (1550 mg) was dissolved in water (100 ml) and the pH corrected to 3.2-3.8 with hydrochloric acid at room temperature. The solution was then warmed to 60° C.-65° C. for 1 hours and then the solution was cooled to 50-60° C. At the same time, the pH was corrected to 4.5-5.0 with sodium hydroxide. Then the solution was quickly frozen and lyophilized. The lyophilized epirubicin hydrochloride obtained (1490 mg) showed 99.7% purity (containing 0.1% dimer and 0.12% aglycone).

### Example 42

### Preparation of Stable Lyophilized Idarubicin HCl

Idarubicin hydrochloride (5.0 g) was dissolved in water (500 ml). Then 58 microlitres of AMBERLITE® IRA-67 (1.6M) acetate (meaning 1.0% molar amount with respect to the molar amount of idarubicin hydrochloride) and 4.5 mg of acetic acid (meaning 0.8% molar amount with respect to the molar amount of idarubicin hydrochloride) were added. The suspension was slurried for two hours, then filtered, frozen and lyophilized. The lyophilized idarubicin hydrochloride obtained (4.95 g) showed 99.9% purity (containing 0.09% aglycone).

### Example 43

### Preparation of Stable Lyophilized Doxorubicin HCl

Doxorubicin hydrochloride (5.0 g) was dissolved in water (200 ml) and the pH corrected to 4.6-4.8 with IN hydrochloric acid at room temperature. Then 80 microlitres of AMBERLITE® IRA-67 (1.6M) acetate (meaning 1.5% molar amount with respect to the molar amount of doxorubicin hydrochloride) was added. The solution was then filtered, frozen and lyophilized. The lyophilized doxorubicin hydrochloride obtained (4.95 g) showed 99.8% purity (containing 0.10% aglycone).

### Example 44

### Preparation of Stable Lyophilized Epirubicin HCl

Epirubicin hydrochloride (1550 mg) was dissolved in water (100 ml) and the pH corrected to 3.2-3.8 with hydrochloric acid at room temperature. The solution was warmed to 60° C.-65° C. for 1 hour and then the solution was cooled to 50-60° C. At the same time, the pH was corrected to 4.5-5.0 with AMBERLITE® IRA-67 free base. Then the solution was quickly filtered, frozen, and lyophilized. The lyophilized epirubicin hydrochloride obtained (1493 mg) showed 99.7% purity (containing 0.10% dimer and 0.10% aglycone).

1. A lyophilized anthracycline glycoside salt, wherein the anthracycline glycoside is selected from the group consisting of Epirubicin, Idarubicin, Epidaunorubicin, and Daunorubicin.

2. A stable lyophilized anthracycline glycoside salt, wherein the anthracycline glycoside is selected from the group consisting of Epirubicin, Idarubicin, Epidaunorubicin, and Daunorubicin.

3. The lyophilized anthracycline glycoside salt according to claim 1, wherein the anthracycline glycoside is either Idarubicin or Epirubicin.

4. The lyophilized anthracycline glycoside salt according to claim 1, wherein the salt is selected from the group consisting of hydrochloride (HCl), hydrobromide (HBr), emi-sulphate (HSO4-), and salts of organic bicarboxylic acids.

5. The lyophilized anthracycline glycoside salt according to claim 4, wherein the organic bicarboxylic acid is selected from the group consisting of maleic acid, succinic acid, glutaric acid and formic acid.

6. The lyophilized anthracycline glycoside salt according to claim 4, wherein the salt is a hydrochloride salt.

7. A method of stabilizing an anthracycline glycoside salt comprising combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether;
wherein the anthracycline glycoside salt is selected from the group consisting of an Epirubicin salt, an Idarubicin salt, an Epidaunorubicin salt, and a Daunorubicin salt.

8. The method according to claim 7, wherein the anthracycline glycoside salts are selected from a group consisting of: anthracycline glycoside hydrochloride, anthracycline glycoside hydrogenbromide, anthracycline glycoside emi-sulphate, and anthracycline glycoside salts of organic bicarboxylic acids.

9. The method according to claim 8, wherein the organic bicarboxylic acid is selected from the group consisting of: maleic acid, succinic acid, glutaric acid and formic acid.

10. The method according to claim 8, wherein the anthracycline glycoside salt is a hydrochloride salt.

11. The method according to claim 7, wherein the anthracycline glycoside salt contains traces of free acid.

12. The method according to claim 11, wherein the acid is HCl.

13. The method according to claim 7, wherein the solid anthracycline glycoside salt is either crystalline or amorphous.

14. The method according to claim 11, wherein the solid anthracycline glycoside salt is crystalline.

15. The method according to claim 7, wherein the alcohol solvent is methanol, ethanol or isopropanol.

16. The method according to claim 7, wherein the ketone solvent is acetone.

17. The method according to claim 7, wherein the ether solvent is tetrahydrofuran, 1,2-dimethoxymethane or 2-methoxyethanol.

18. The method according to claim 7, wherein the solvent is water.

19. The method according to claim 7, wherein the solid anthracycline glycoside salts are combined with the solvent to obtain a solution, prior to the addition of the buffer.

20. The method according to claim 7, wherein the buffer comprises a salt derived from mixing a weak base and a weak acid or is a mixture of a salt, derived from mixing a weak base and a weak acid, with a weak acid.

21. The method according to claim 20, wherein the salt is selected from the group consisting of: ammonium acetate, ammonium formate, ammonium hydrogencarbonate and sodium hydrogencarbonate.

22. The method according to claim 20, wherein the weak acid combined with the salt is selected from the group consisting of acetic acid, formic acid and H₂CO₃.

23. The method according to claim 20, wherein the mixture of a salt and a weak acid is selected from the group consisting of a mixture of ammonium acetate and acetic acid, a mixture of ammonium formate and acetic acid, a mixture of ammonium formate and formic acid, a mixture of ammonium hydrogencarbonate and H₂CO₃, and a mixture of sodium hydrogencarbonate and H₂CO₃.

24. The method according to claim 7, wherein the starting anthracycline glycoside salt is an Idarubicin salt and the buffer comprises a mixture of ammonium acetate and acetic acid, or a mixture of ammonium hydrogencarbonate and H₂CO₃.

25. The method according to claim 7, wherein the starting anthracycline glycoside salt is an Epirubicin salt, and the buffer is ammonium acetate.

26. The method according to claim 7, wherein the starting anthracycline glycoside salt is a Doxorubicin salt, and the buffer is ammonium acetate, a mixture of ammonium formate and acetic acid, a mixture of ammonium formate and formic acid, or a mixture of sodium hydrogencarbonate and H₂CO₃.

27. The method according to claim 7, wherein the starting anthracycline glycoside salt is Idarubicin or Epirubicin salts and the buffer is ammonium hydrogencarbonate.

28. The method according to claim 7, wherein the buffer is used in an amount of 0.5% to about 1.5% mole equivalent per mole equivalent of the starting anthracycline glycoside salt.

29. The method according to claim 7, wherein the anthracycline glycoside salt is an Epirubicin salt the method further comprises, prior to the step of combining the anthracycline glycoside salt, a solvent and a buffer, steps of purifying the Epirubicin salt comprising
a) combining the anthracycline glycoside salt containing an alpha hydroxyl ketone moiety with a solvent selected from the group consisting of water, and mixtures of water with alcohol, ketone or ether;
b) heating the mixture for a short period of time; and
c) quickly cooling the heated mixture.

30. The method according to claim 29, wherein the mixture is heated to a temperature of about 50° C. to about 70° C. for a period of time of about 30 to about 90 minutes and wherein the mixture is cooled to room temperature.

31. The method according to claim 30, wherein the period of time for heating the mixture is about 30 to about 60 minutes.

32. The method according to claim 29, wherein when the pH of the starting mixture is at least 5.4, the pH is adjusted to about 3.2 to about 3.8 by adding an acid to the solution, prior to heating it.

33. The method according to claim 32, wherein the acid is selected from the group consisting of hydrogenchloride (HCl), hydrogenbromide (HBr), sulphuric acid bicarboxylic acids like maleic acid, succinic acid, glutaric acid or strong organic acid like formic acid.

34. The method according to claim 33, wherein the acid is HCl.

35. A method for stabilizing an anthracycline glycoside salt comprising:
a) combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether; and
b) filtering the obtained mixture;
wherein the resin is an organic co-polymer having a basic nature, and the anthracycline glycoside salt is selected from the group consisting of an Epirubicin salt, an Idarubicin salt, an Epidaunorubicin salt, a Doxorubicin salt and a Daunorubicin salt.

36. The method according to claim 35, wherein the anthracycline glycoside salts are selected from a group consisting of: anthracycline glycoside hydrochloride, anthracycline glycoside hydrogenbromide, anthracycline glycoside emi-sulphate, and anthracycline glycoside salts of organic bicarboxylic acids.

37. The method according to claim 36, wherein the organic bicarboxylic acid is selected from the group consisting of: maleic acid, succinic acid, glutaric acid and formic acid.

38. The method according to claim 36, wherein the anthracycline glycoside salt is a hydrochloride salt.

39. The method according to claim 35, wherein the anthracycline glycoside salt contains traces of free acid.

40. The method according to claim 39, wherein the acid is HCl.

41. The method according to claim 35, wherein the solid anthracycline glycoside salt is either crystalline or amorphous.

42. The method according to claim 39, wherein the solid anthracycline glycoside salt is crystalline.

43. The method according to claim 35, wherein the alcohol solvent is methanol, ethanol or isopropanol.

44. The method according to claim 35, wherein the ketone solvent is acetone.

45. The method according to claim 35, wherein the ether solvent is tetrahydrofuran, 1,2-dimethoxymethane or 2-methoxyethanol.

46. The method according to claim 35, wherein the solvent is water.

47. The method according to claim 35, wherein the solid anthracycline glycoside salts are combined with the solvent to obtain a solution, prior to the addition of the resin.

48. The method according to claim 35, wherein the resin comprises a tertiary amine linked to a solid surface or a salt of this teriary amine.

49. The method according to claim 48, wherein the salt is a weak acid salt.

50. The method according to claim 49, wherein the weak acid is either acetic acid or formic acid.

51. The method according to claim 48, wherein the resin is selected from a group consisting of: Amberlite®, Amberlite: FPA51, Amberlite FPA53, Amberlite FPA54, Amberlite FPA55, Amberlite FPA40, Amberlite FPA42, Amberlite FPA90, Amberlite FPA91, Amberlite FPA97, Amberlite FPA98, Amberlite IRA900, Amberlite IRA910, Amberjet 4200, Amberlite IRA 67, Amberlite IRA 96, Amberlyst A21, Amberlyst A23, and Amberlyst A24.

52. The method according to claim 48, wherein the resin is Amberlite®.

53. The method according to claim 48, wherein the Amberlite® is Amberlite® Ira-67.

54. The method according to claim 35, wherein the starting anthracycline glycoside salt is either a Doxorubicin or an Idarubicin salt, and the resin is AMBERLITE® IRA-67 acetate.

55. The method according to claim 35, wherein the starting anthracycline glycoside salt is an Epirubicin salt, and the resin is AMBERLITE® IRA-67 free base.

56. The method according to claim 35, wherein the resin is used in an amount of 0.5% to about 1.5% mole equivalent per mole equivalent of the starting anthracycline glycoside salt.

57. The method according to claim 35, wherein the anthracycline glycoside salt contains an alpha hydroxyl ketone moiety and wherein prior to the step of combining the anthracycline glycoside salt, a solvent and resin, the method further comprises the steps of purifying the anthracycline glycoside salt containing an alpha hydroxyl ketone moiety comprising
a) combining the anthracycline glycoside salt containing an alpha hydroxyl ketone moiety with a solvent selected from the group consisting of water, and mixtures of water with alcohol, ketone or ether;
b) heating the mixture for a short period of time; and
c) quickly cooling the heated mixture.

58. The method according to claim 57, wherein the mixture is heated to a temperature of about 50° C. to about 70° C. for a period of time of about 30 to about 90 minutes and wherein the mixture is cooled to room temperature.

59. The method according to claim 58, wherein the period of time for heating the mixture is about 30 to about 60 minutes.

60. The method according to claim 57, wherein when the pH of the starting mixture is at least 5.4, the pH is adjusted to about 3.2 to about 3.8 by adding an acid to the solution, prior to heating it.

61. The method according to claim 60, wherein the acid is selected from the group consisting of hydrogenchloride (HCl), hydrogenbromide (HBr), sulphuric acid bicarboxylic acids like maleic acid, succinic acid, glutaric acid or strong organic acid like formic acid.

62. The method according to claim 61, wherein the acid is HCl.

63. The method according to claim 57, wherein the anthracycline glycoside is selected from the group consisting of Epirubicin and Doxorubicin.

64. A method of preparing stable lyophilized forms of anthracycline glycoside salts, comprising:
a) combining a solid anthracycline glycoside salt, about 0.3% to about 3% mole equivalent of a buffer per mole equivalent of the anthracycline glycoside salt, and a solvent selected from the group consisting of water, and mixtures of water with alcohol, ketone or ether;
b) freezing, and
c) lyophilizing the obtained mixture; wherein the anthracycline glycoside salt is selected from the group consisting of an Epirubicin salt, an Idarubicin salt, an Epidaunorubicin salt, and a Daunorubicin salt.

65. The method according to claim 64, wherein the anthracycline glycoside salts are selected from a group consisting of: anthracycline glycoside hydrochloride, anthracycline glycoside hydrogenbromide, anthracycline glycoside emi-sulphate, and anthracycline glycoside salts of organic bicarboxylic acids.

66. The method according to claim 65, wherein the organic bicarboxylic acid is selected from the group consisting of: maleic acid, succinic acid, glutaric acid and formic acid.

67. The method according to claim 65, wherein the anthracycline glycoside salt is a hydrochloride salt.

68. The method according to claim 64, wherein the anthracycline glycoside salt contains traces of free acid.

69. The method according to claim 68, wherein the acid is HCl.

70. The method according to claim 64, wherein the solid anthracycline glycoside salt is either crystalline or amorphous.

71. The method according to claim 68, wherein the solid anthracycline glycoside salt is crystalline.

72. The method according to claim 65, wherein the alcohol solvent is methanol, ethanol or isopropanol.

73. The method according to claim 65, wherein the ketone solvent is acetone.

74. The method according to claim 65, wherein the ether solvent is tetrahydrofuran, 1,2-dimethoxymethane or 2-methoxyethanol.

75. The method according to claim 65, wherein the solvent is water.

76. The method according to claim 65, wherein the solid anthracycline glycoside salts are combined with the solvent to obtain a solution, prior to the addition of the buffer.

77. The method according to claim 65, wherein the buffer comprises a salt derived from mixing a weak base and a weak acid or is a mixture of a salt, derived from mixing a weak base and a weak acid, with a weak acid.

78. The method according to claim 77, wherein the salt is selected from the group consisting of: ammonium acetate, ammonium formate, ammonium hydrogencarbonate and sodium hydrogencarbonate.

79. The method according to claim 77, wherein the weak acid combined with the salt is selected from the group consisting of acetic acid, formic acid and H₂CO₃.

80. The method according to claim 77, wherein the mixture of a salt and a weak acid is selected from the group consisting of a mixture of ammonium acetate and acetic acid, a mixture of ammonium formate and acetic acid, a mixture of ammonium formate and formic acid, a mixture of ammonium hydrogencarbonate and H₂CO₃, and a mixture of sodium hydrogencarbonate and H₂CO₃.

81. The method according to claim 65, wherein the starting anthracycline glycoside salt is an Idarubicin salt and the buffer comprises a mixture of ammonium acetate and acetic acid, or a mixture of ammonium hydrogencarbonate and H₂CO₃.

82. The method according to claim 65, wherein the starting anthracycline glycoside salt is an Epirubicin salt, and the buffer is ammonium acetate.

83. The method according to claim 65, wherein the starting anthracycline glycoside salt is Idarubicin or Epirubicin salts and the buffer is ammonium hydrogencarbonate.

85. (Canceled).

86. The method according to claim 65, wherein the buffer is used in an amount of 0.5% to about 1.5% mole equivalent per mole equivalent of the starting anthracycline glycoside salt.

87. The method according to claim 65, wherein the anthracycline glycoside salt is an Epirubicin salt the method further comprises, prior to the step of combining the anthracycline glycoside salt, a solvent and a buffer, steps of purifying the Epirubicin salt comprising
a) combining the anthracycline glycoside salt containing an alpha hydroxyl ketone moiety with a solvent selected from the group consisting of water, and mixtures of water with alcohol, ketone or ether;
b) heating the mixture for a short period of time; and
c) quickly cooling the heated mixture.

88. The method according to claim 87, wherein the mixture is heated to a temperature of about 50° C. to about 70° C. for a period of time of about 30 to about 90 minutes and wherein the mixture is cooled to room temperature.

89. The method according to claim 88, wherein the period of time for heating the mixture is about 30 to about 60 minutes.

90. The method according to claim 87, wherein when the pH of the starting mixture is at least 5.4, the pH is adjusted to about 3.2 to about 3.8 by adding an acid to the solution, prior to heating it.

91. The method according to claim 90, wherein the acid is selected from the group consisting of hydrogenchloride (HCl), hydrogenbromide (HBr), sulphuric acid bicarboxylic acids like maleic acid, succinic acid, glutaric acid or strong organic acid like formic acid.

92. The method according to claim 91, wherein the acid is HCl.

93. A method of preparing stable lyophilized forms of anthracycline glycoside salt, comprising:
a) combining a solid anthracycline glycoside salt, about 0.3% to about 3% mole equivalent of a resin per mole equivalent of the anthracycline glycoside salt, and a solvent selected from the group consisting of water, and mixtures of water with alcohol, ketone or ether;
b) filtering the obtained mixture;
c) freezing, and
d) lyophilizing the filtrate;
wherein the resin is an organic co-polymer having a basic nature, and the anthracycline glycoside salt is selected from the group consisting of an Epirubicin salt, an Idarubicin salt, an Epidaunorubicin salt, a Doxorubicin salt and a Daunorubicin salt.

94. The method according to claim 93, wherein the anthracycline glycoside salts are selected from a group consisting of: anthracycline glycoside hydrochloride, anthracycline glycoside hydrogenbromide, anthracycline glycoside emi-sulphate, and anthracycline glycoside salts of organic bicarboxylic acids.

95. The method according to claim 94, wherein the organic bicarboxylic acid is selected from the group consisting of: maleic acid, succinic acid, glutaric acid and formic acid.

96. The method according to claim 94, wherein the anthracycline glycoside salt is a hydrochloride salt.

97. The method according to claim 93, wherein the anthracycline glycoside salt contains traces of free acid.

98. The method according to claim 97, wherein the acid is HCl.

99. The method according to claim 93, wherein the solid anthracycline glycoside salt is either crystalline or amorphous.

100. The method according to claim 97, wherein the solid anthracycline glycoside salt is crystalline.

101. The method according to claim 93, wherein the alcohol solvent is methanol, ethanol or isopropanol.

102. The method according to claim 93, wherein the ketone solvent is acetone.

103. The method according to claim 93, wherein the ether solvent is tetrahydrofuran, 1,2-dimethoxymethane or 2-methoxyethanol.

104. The method according to claim 93, wherein the solvent is water.

105. The method according to claim 93, wherein the solid anthracycline glycoside salts are combined with the solvent to obtain a solution, prior to the addition of the resin.

106. The method according to claim 93, wherein the resin comprises a tertiary amine linked to a solid surface or a salt of this teriary amine.

107. The method according to claim 106, wherein the salt is a weak acid salt.

108. The method according to claim 107, wherein the weak acid is either acetic acid or formic acid.

109. The method according to claim 106, wherein the resin is selected from a group consisting of: Amberlite®, Amberlite: FPA51, Amberlite FPA53, Amberlite FPA54, Amberlite FPA55, Amberlite FPA40, Amberlite FPA42, Amberlite FPA90, Amberlite FPA91, Amberlite FPA97, Amberlite FPA98, Amberlite IRA900, Amberlite IRA910, Amberjet 4200, Amberlite IRA 67, Amberlite IRA 96, Amberlyst A21, Amberlyst A23, and Amberlyst A24.

110. The method according to claim 106, wherein the resin is Amberlite®.

111. The method according to claim 106, wherein the Amberlite® is Amberlite® Ira-67.

112. The method according to claim 93, wherein the starting anthracycline glycoside salt is either a Doxorubicin or an Idarubicin salt, and the resin is AMBERLITE® IRA-67 acetate.

113. The method according to claim 93, wherein the starting anthracycline glycoside salt is an Epirubicin salt, and the resin is AMBERLITE® IRA-67 free base.

114. The method according to claim 93, wherein the resin is used in an amount of 0.5% to about 1.5% mole equivalent per mole equivalent of the starting anthracycline glycoside salt.

115. The method according to claim 93, wherein the anthracycline glycoside salt contains an alpha hydroxyl ketone moiety and wherein prior to the step of combining the anthracycline glycoside salt, a solvent and a resin, the process further comprises the steps of purifying the anthracycline glycoside salt containing an alpha hydroxyl ketone moiety comprising
a) combining the anthracycline glycoside salt containing an alpha hydroxyl ketone moiety with a solvent selected from the group consisting of water, and mixtures of water with alcohol, ketone or ether;
b) heating the mixture for a short period of time; and
c) quickly cooling the heated mixture.

116. The method according to claim 115, wherein the mixture is heated to a temperature of about 50° C. to about 70° C. for a period of time of about 30 to about 90 minutes and wherein the mixture is cooled to room temperature.

117. The method according to claim 116, wherein the period of time for heating the mixture is about 30 to about 60 minutes.

118. The method according to claim 116, wherein when the pH of the starting mixture is at least 5.4, the pH is adjusted to about 3.2 to about 3.8 by adding an acid to the solution, prior to heating it.

119. The method according to claim 118, wherein the acid is selected from the group consisting of hydrogenchloride (HCl), hydrogenbromide (HBr), sulphuric acid bicarboxylic acids like maleic acid, succinic acid, glutaric acid or strong organic acid like formic acid.

120. The method according to claim 119, wherein the acid is HCl.

121. The method according to claim 115, wherein the anthracycline glycoside is selected from the group consisting of Epirubicin and Doxorubicin.

122. A pharmaceutical formulation comprising stable lyophilized anthracycline glycoside hydrochloride of claim 2, and pharmaceutically acceptable excipients.

123. The stable lyophilized anthracycline glycoside salt according to claim 2, wherein the anthracycline glycoside is either Idarubicin or Epirubicin.

124. The stable lyophilized anthracycline glycoside salt according to claim 2, wherein the salt is selected from the group consisting of hydrochloride (HCl), hydrobromide (HBr), emi-sulphate (HSO4-), and salts of organic bicarboxylic acids.

125. The lyophilized anthracycline glycoside salt according to claim 124, wherein the organic bicarboxylic acid is selected from the group consisting of maleic acid, succinic acid, glutaric acid and formic acid.

126. The lyophilized anthracycline glycoside salt according to claim 124, wherein the salt is a hydrochloride salt.
